(19) United States
(12) Patent Application Publication
(10) Pub. No.: US 2024/0315506 A1
(43) Pub. Date: Sep. 26, 2024

(54) CLEANER AND METHOD FOR CONTROLLING THEREOF

(71) Applicants: SAMSUNG ELECTRONICS CO., LTD., Suwon-si (KR); MEDIAEVER.CO., LTD., Suwon-si (KR)

(72) Inventors: Yeona HWANG, Suwon-si (KR); Sujin SEONG, Suwon-si (KR); Kyuho SHIN, Suwon-si (KR); Byung-In MA, Suwon-si (KR); Yong Hoon LEE, Suwon-si (KR); Hyeongjoon SEO, Suwon-si (KR); Kisup LEE, Suwon-si (KR)

(73) Assignees: SAMSUNG ELECTRONICS CO., LTD., Suwon-si (KR); MEDIAEVER.CO., LTD., Suwon-si (KR)

(21) Appl. No.: 18/731,950

(22) Filed: Jun. 3, 2024

Related U.S. Application Data
(63) Continuation of application No. PCT/KR2024/000496, filed on Jan. 10, 2024.

(30) Foreign Application Priority Data
Feb. 17, 2023 (KR) 10-2023-0021568

(51) Int. Cl. A47L 9/28; A47L 9/30

(52) U.S. Cl. CPC A47L 9/2815; A47L 9/2842; A47L 9/30

(57) ABSTRACT

The disclosure provides a cleaner and a controlling method thereof. A cleaner according to an embodiment of the disclosure includes a suction part, a suction device which generates airflow such that dust is suctioned through the suction part, a detection device which is arranged on one side of the suction part, and emits a light of a predetermined wavelength, and detects a wavelength of a partial light emitted, and at least one processor which controls the suction device, wherein the at least one processor is configured to control the detection device to emit a light of a predetermined wavelength during an operation of the suction device, and detect whether allergen substances exist in the dust suctioned through the suction part based on a wavelength of a reflective light detected in the detection device.

### CROSS-REFERENCE TO RELATED APPLICATION

This application is a bypass continuation of International Application No. PCT/KR2024/000496, filed on Jan. 10, 2024, which is based on and claims priority to Korean Patent Application No. 10-2023-0021568, filed on Feb. 17, 2023, in the Korean Intellectual Property Office, the disclosures of which are incorporated by reference herein in their entireties.

### BACKGROUND

### 1. Field

The disclosure relates to a cleaner and a method for controlling thereof, and more particularly, to a cleaner that detects allergen substances included in dust, and a method for controlling thereof.

### 2. Description of Related Art

As electronic technologies have developed, cleaners in various forms from conventional stick type and canister type cleaners to robot cleaners have been released, and services provided by cleaners to users have also become various. As an example, in the case of a robot cleaner, it became possible to control the robot cleaner through a user's voice or a user's terminal device, and information on a location wherein the robot cleaner performs a cleaning operation and a location wherein the cleaning operation was completed is provided to a user in real time.

Recently, a function of detecting allergen substances inside dust suctioned by a cleaner in a cleaning process of the cleaner and providing related information of allergen substances (e.g., tick alert warning, etc.) to a user is occasionally mounted on a cleaner. Meanwhile, in the past, it was identified whether allergen substances existed in the dust based on the sizes of the allergen substances. However, according to such a method, a problem that dust, foreign substances, etc. having big sizes in the dust suctioned by a cleaner are identified as allergen substances wrongly, and wrong information is provided to a user is generated.

### SUMMARY

A cleaner according to an embodiment of the disclosure includes a suction part, a suction device which generates airflow such that dust is suctioned through the suction part, a detection device which is arranged on one side of the suction part, and emits a light of a predetermined wavelength, and detects a wavelength of a partial light emitted, and at least one processor which controls the suction device. The at least one processor is configured to control the detection device to emit a light of a predetermined wavelength during an operation of the suction device, and detect whether allergen substances exist in the dust suctioned through the suction part based on a wavelength of a reflective light detected in the detection device.

Here, the at least one processor may identify the amount of the allergen substances based on the strength of the detected light, identify a concentration level of the allergen substances corresponding to the identified amount of the allergen substances, and control the suction device to generate the airflow in suction strength corresponding to the identified concentration level.

Here, the cleaner may further include an output interface including a plurality of light emitting elements, and the at least one processor may control the plurality of light emitting elements such that a light of a color corresponding to the identified concentration level is emitted.

Also, the cleaner may further include a communication interface, and a memory storing map data corresponding to a space wherein the cleaner is located, and the at least one processor may identify the amount of allergen substances identified in a plurality of areas of the space based on the amount of the allergen substances, display information on the amount of the allergen substances corresponding to each area on the map data and store it in the memory, periodically update the stored map data based on the amount of the allergen substances, and transmit the updated map data to a user terminal device through the communication interface.

In addition, the cleaner may further include a collection part collecting the suctioned dust which includes at least one heating coil, and the at least one processor may, based on the amount of the allergen substances being greater than or equal to a predetermined value, provide power to the at least one heating coil such that the allergen substances are inactivated.

Meanwhile, the predetermined wavelength may be from 350 nm to 405 nm, and the at least one processor may, based on the wavelength of the reflected light being from 220 nm to 500 nm, identify that the allergen substances exist.

The detection device may include a chamber into which the dust is introduced and of which inside is implemented as a reflector, a light entrance into which an incident light enters, a chamber body including a first light outlet and a second light outlet for injecting an incident light irradiated on the dust, a light emitting part which irradiates the incident light to the light entrance, and blocks ambient lights introduced into the incident light, a light receiving part which delivers a radiant light injected from the first light outlet by separating the light into a first path and a second path, detects a scattering light from the radiant light transmitted to the first path, and blocks the ambient lights introduced into the radiant light transmitted to the second path and detects the reflected light, and a diffused reflection reduction part which is coupled in a predetermined location from the first light outlet, and reduces a diffused reflective light injected from the first light outlet as an opening is formed in the center part.

Here, the suction unit may include a suction pipe which provides a suction flow path of the suctioned dust, and the suction pipe may include a filter arranged on one end, and the filter may include a first hole corresponding to the center area of the suction flow path, and in the chamber, dust filtered through the suction pipe may be introduced.

A controlling method of a cleaner according to an embodiment of the disclosure may include the step of controlling the detection device to suction dust. Also, the controlling method may include the step of detecting whether allergen substances exist in the suctioned dust. Here, the suctioning step may include the steps of emitting a light of a predetermined wavelength to the suctioned dust during an operation of the suction device, and collecting the light emitted from the dust. Also, the detecting step may include the step of detecting whether allergen substances exist in the dust based on a wavelength of the collected reflective light.

Here, the detecting step may include the step of identifying the strength of the reflective light, and identifying the amount of the allergen substances based on the identified strength of the reflective light, and the controlling method may further include the steps of identifying a concentration level of the allergen substances corresponding to the identified amount of the allergen substances, and controlling the suction device to generate an airflow in suction strength corresponding to the identified concentration level.

Here, the controlling method may further include the step of controlling a plurality of light emitting elements such that a light of a color corresponding to the identified concentration level is emitted.

Also, the controlling method may include the step of identifying the amount of allergen substances identified in a plurality of areas of a space wherein the cleaner is located based on the amount of the allergen substances, displaying information on the amount of the allergen substances corresponding to each area on map data corresponding to the space and storing it in the memory, identifying the amount of the allergen substances whenever the suction device operates, and periodically updating the stored map data based on the identified amount of the allergen substances, and transmitting the updated map data to a user terminal device.

In addition, the controlling method may include the step of, based on the amount of the allergen substances being greater than or equal to a predetermined value, providing power to at least one heating coil included in a collection part such that the allergen substances are inactivated.

Meanwhile, the predetermined wavelength may be from 350 nm to 405 nm, and in the detecting step, based on the wavelength of the reflected light being from 220 nm to 500 nm, it may be identified that the allergen substances exist.

Also, the cleaner may include a detection device, and the detection device may include a chamber into which the dust is introduced and of which inside is implemented as a reflector, a light entrance into which an incident light enters, a chamber body including a first light outlet and a second light outlet for injecting an incident light irradiated on the dust, a light emitting part which irradiates the incident light to the light entrance, and blocks ambient lights introduced into the incident light, a light receiving part which delivers a radiant light injected from the first light outlet by separating the light into a first path and a second path, detects a scattering light from the radiant light transmitted to the first path, and blocks the ambient lights introduced into the radiant light transmitted to the second path and detects the reflected light, and a diffused reflection reduction part which is coupled in a predetermined location from the first light outlet, and reduces a diffused reflective light injected from the first light outlet as an opening is formed in the center part.

In addition, the cleaner may further include a suction part, and the suction part may include a suction pipe which provides a suction flow path of the suctioned dust, and the suction pipe may include a filter arranged on one end, and the filter may include a first hole corresponding to the center area of the suction flow path, and in the chamber, dust filtered through the suction pipe may be introduced.

Meanwhile, in a non-transitory computer-readable recording medium storing computer instructions for making the cleaner perform operations in case the instructions are executed by a processor of the cleaner, the operations may include the step of controlling the detection device to suction dust. Also, the controlling method may include the step of detecting whether allergen substances exist in the suctioned dust. Here, the suctioning step may include the steps of emitting a light of a predetermined wavelength to the suctioned dust during an operation of the suction device, and collecting the light emitted from the dust. Also, the detecting step may include the step of detecting whether allergen substances exist in the suctioned dust based on a wavelength of the collected reflective light.

### BRIEF DESCRIPTION OF THE DRAWINGS

FIG. 1 is a schematic exemplary diagram of a cleaner according to an embodiment of the disclosure;

FIG. 2 is a schematic block diagram of a cleaner according to an embodiment of the disclosure;

FIG. 3 is an exemplary diagram illustrating a detection device arranged inside a cleaner according to an embodiment of the disclosure;

FIG. 4 is a schematic block diagram of a detection device according to an embodiment of the disclosure;

FIG. 5 is a cross-sectional view illustrating a chamber of a detection device according to an embodiment of the disclosure;

FIG. 6 is a diagram illustrating a relation between a circular mirror and an elliptical mirror inside a chamber of a detection device according to an embodiment of the disclosure;

FIG. 7 is a diagram illustrating a form of instruction of air suctioned into a detection device according to an embodiment of the disclosure;

FIG. 8 is an exemplary diagram illustrating transmission of information regarding allergen substances to a user terminal through a communication interface according to an embodiment of the disclosure;

FIG. 9 is an exemplary diagram illustrating control of a plurality of light emitting elements such that a light of a color corresponding to the concentration of allergen substances is emitted according to an embodiment of the disclosure;

FIG. 10 is a detailed configuration diagram of a cleaner according to an embodiment of the disclosure;

FIG. 11 is an exemplary diagram illustrating transmission by a robot cleaner of information regarding allergen substances to a user terminal through a communication interface according to an embodiment of the disclosure; and

FIG. 12 is a flow chart of a controlling method of a cleaner according to an embodiment of the disclosure.

### DETAILED DESCRIPTION

Various modifications may be made to the embodiments of the disclosure, and there may be various types of embodiments. Accordingly, specific embodiments will be illustrated in drawings, and the embodiments will be described in detail in the detailed description. However, it should be noted that the various embodiments are not for limiting the scope of the disclosure to a specific embodiment, but they should be interpreted to include various modifications, equivalents, and/or alternatives of the embodiments of the disclosure. Also, with respect to the detailed description of the drawings, similar components may be designated by similar reference numerals.

Also, in describing the disclosure, in case it is determined that detailed explanation of related known functions or components may unnecessarily confuse the gist of the disclosure, the detailed explanation in that regard will be omitted.

In addition, the embodiments below may be modified in various different forms, and the scope of the technical idea of the disclosure is not limited to the embodiments below. Rather, these embodiments are provided to make the disclosure more sufficient and complete, and to fully convey the technical idea of the disclosure to those skilled in the art.

Further, the terms used in the disclosure are used just to explain specific embodiments, and are not intended to limit the scope of the disclosure. In addition, singular expressions include plural expressions, unless defined obviously differently in the context.

Also, in the disclosure, expressions such as “have,” “may have,” “include,” and “may include” denote the existence of such characteristics (e.g.: elements such as numbers, functions, operations, and components), and do not exclude the existence of additional characteristics.

In addition, in the disclosure, the expressions “A or B,” “at least one of A and/or B,” or “one or more of A and/or B” and the like may include all possible combinations of the listed items. For example, “A or B,” “at least one of A and B,” or “at least one of A or B” may refer to all of the following cases: (1) including at least one A, (2) including at least one B, or (3) including at least one A and at least one B.

Further, the expressions “first,” “second,” and the like used in the disclosure may describe various elements regardless of any order and/or degree of importance. Also, such expressions are used only to distinguish one element from another element, and are not intended to limit the elements.

Meanwhile, the description in the disclosure that one element (e.g.: a first element) is “(operatively or communicatively) coupled with/to” or “connected to” another element (e.g.: a second element) should be interpreted to include both the case where the one element is directly coupled to the another element, and the case where the one element is coupled to the another element through still another element (e.g.: a third element).

In contrast, the description that one element (e.g.: a first element) is “directly coupled” or “directly connected” to another element (e.g.: a second element) can be interpreted to mean that still another element (e.g.: a third element) does not exist between the one element and the another element.

Also, the expression “configured to” used in the disclosure may be interchangeably used with other expressions such as “suitable for,” “having the capacity to,” “designed to,” “adapted to,” “made to,” and “capable of,” depending on cases. Meanwhile, the term “configured to” does not necessarily mean that a device is “specifically designed to” in terms of hardware.

Instead, under some circumstances, the expression “a device configured to” may mean that the device “is capable of” performing an operation together with another device or component. For example, the phrase “a processor configured to perform A, B, and C” may mean a dedicated processor (e.g.: an embedded processor) for performing the corresponding operations, or a generic-purpose processor (e.g.: a CPU or an application processor) that can perform the corresponding operations by executing one or more software programs stored in a memory device.

Further, in the embodiments of the disclosure, ‘a module’ or ‘a unit’ may perform at least one function or operation, and may be implemented as hardware or software, or as a combination of hardware and software. Also, a plurality of ‘modules’ or ‘units’ may be integrated into at least one module and implemented as at least one processor, excluding ‘a module’ or ‘a unit’ that needs to be implemented as specific hardware.

Meanwhile, various elements and areas in the drawings were illustrated schematically. Accordingly, the technical idea of the disclosure is not limited by the relative sizes or intervals illustrated in the accompanying drawings.

Hereinafter, the embodiments according to the disclosure will be described in detail with reference to the accompanying drawings, such that those having ordinary skill in the art to which the disclosure belongs can easily carry out the disclosure.

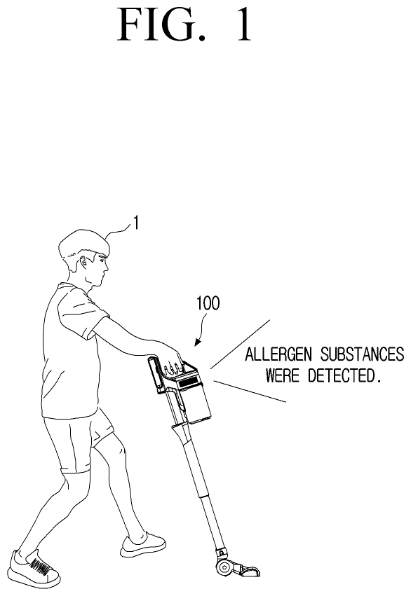

FIG. 1 is a schematic exemplary diagram of a cleaner 100 according to an embodiment of the disclosure.

The cleaner 100 according to an embodiment of the disclosure identifies whether allergen substances exist in a process of performing a cleaning operation. More specifically, in a process of suctioning air, dust, etc. through a suction part of the cleaner 100, the cleaner 100 identifies whether allergen substances exist in the suctioned dust. Then, if it is identified that allergen substances exist, the cleaner 100 may output information indicating that allergen substances exist to the user. By this, the user can recognize that allergen substances exist in the home (e.g., a space such as a house, a building, etc. wherein the user is located). Meanwhile, allergen substances include pollen, fungi, ticks, and other microorganisms, etc., and refer to substances that cause allergic reactions to a user.

As allergen substances cause allergies of human bodies, it is important to correctly and immediately notify to a user that allergen substances exist. Meanwhile, in the case of a conventional cleaner 100, the cleaner 100 identified allergen substances in dust by collecting (or capturing) the suctioned dust (or, foreign substances, trash, etc.), and then identifying the size of the collected dust. For example, the conventional cleaner 100 identified substances in sizes from 2 μm to 300 μm inside dust as allergen substances.

However, in this case, as cases wherein dust and foreign substances having sizes within the aforementioned size range were identified wrongly as allergen substances occurred, a problem that the cleaner 100 provided wrong information to a user was generated. In addition, as the cleaner 100 identifies allergen substances after collecting the suctioned dust, etc. as described above, it is difficult to immediately notify a location wherein allergen substances exist in a home to a user. Due to this, it was difficult for a user to correctly identify in which location or area inside the home allergen substances existed, and it was also difficult to take a proper measure for allergen substances.

For resolving such a task, as disclosed in FIG. 1, the cleaner 100 according to an embodiment of the disclosure identifies whether allergen substances exist in the dust that is suctioned in real time in a process of performing a cleaning operation. The process of performing a cleaning operation may be a process wherein the cleaner 100 suctions dust by driving the suction device (e.g., a motor, a fan, etc.) of the cleaner 100. The cleaner 100 may detect allergen substances in real time in the process of performing a cleaning operation, and output information indicating that allergen substances were detected. For example, the cleaner 100 may output a voice message indicating that allergen substances were detected. Through this, the user is provided with information indicating allergen substances as soon as allergen substances are detected, and can thus identify the location wherein the allergen substances exist correctly and remove the allergen substances.

Meanwhile, the cleaner 100 according to an embodiment of the disclosure includes not only a cleaner in a form wherein the user performs cleaning by using a handle provided on the cleaner 100 such as a stick type cleaner, a canister type cleaner, etc., but also a robot cleaner that includes a driving part and performs a cleaning operation by itself without the user's operation (or just with the user's control instruction). However, hereinafter, explanation will be described by assuming the cleaner 100 as a stick type cleaner, for the convenience of explanation of the disclosure.

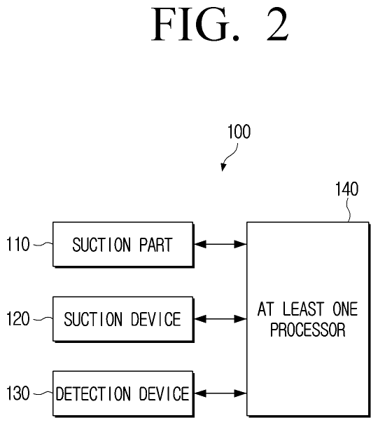

FIG. 2 is a schematic block diagram of the cleaner 100 according to an embodiment of the disclosure.

Referring to FIG. 2, the cleaner 100 includes a suction part 110, a suction device 120, a detection device 130, and at least one processor 140. Meanwhile, in FIG. 2, the suction part 110 and the suction device 120 were illustrated as separate components, but the suction part 110 and the suction device 120 may also be implemented as one component.

The suction part 110 is a component that suctions dust, and may include a brush, a head, a suction pipe, etc. Specifically, the suction part 110 may include a head that performs a role of an entrance through which dust on a surface to be cleaned (a bottom surface, a wall surface, etc. to be cleaned) is suctioned into the cleaner 100 as the suction device 120 is driven, and a suction pipe that provides a suction flow path to the suctioned dust. The brush may be arranged on one surface of the head, and perform a role of separating dust from the surface to be cleaned. Meanwhile, the brush may be implemented as a material that has a small friction coefficient and has good wear resistance such as natural hair or polyamide(PA: nylon), etc.

Here, the suction part 110 may further include a sensor that detects movements of the brush. In particular, the sensor may detect a moving direction and a refracting direction of the brush as the cleaner 100 moves, and also detect the speed, etc. of the brush. The cleaner 100 may identify the frictional force between the bottom surface of the brush and the brush based on a sensing value acquired through the sensor, and identify the material of the bottom surface based on the identified frictional force.

Meanwhile, the suction device 120 may generate a rotational force and generate an airflow such that dust is suctioned into the cleaner 100 through the suction part 110. For this, the suction device 120 may include a motor, a fan, etc. The motor may generate a rotational force and provide the force to the fan, and the fan may generate an airflow based on the rotational force provided from the motor and generate a suction force.

The detection device 130 is arranged on one side of the suction part 110, and emits a light of a predetermined wavelength, and detects a wavelength of a partial light emitted, and detects allergen substances in the dust suctioned through the suction part 110. Specifically, the detection device 130 may be arranged on one side of the suction pipe implementing the suction flow path. Accordingly, the detection device 130 may detect whether allergen substances exist in the dust suctioned in real time in a process wherein the cleaner 100 suctions dust. In particular, the detection device 130 may emit a light of a predetermined wavelength to the suctioned dust. Further, the detection device 130 may acquire a light emitted (or reflected) from the dust based on the emitted light.

In particular, in case allergen substances exist in the dust, the allergen substances may absorb the light emitted from the detection device 130, and then emit a light of another wavelength. Accordingly, the detection device 130 may acquire the light emitted from the allergen substances, and then identify the wavelength of the acquired light, and identify whether allergen substances exist in the suctioned dust based on the identified wavelength.

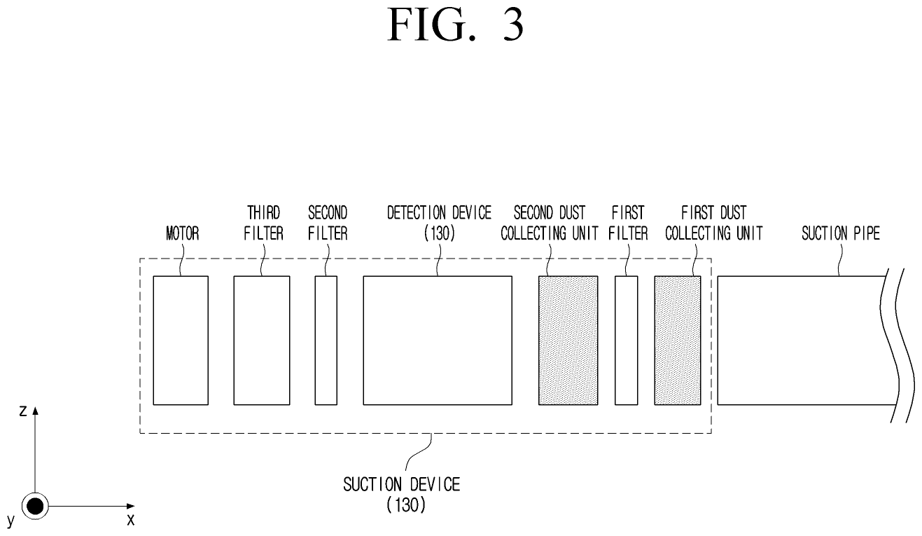

FIG. 3 is an exemplary diagram illustrating the detection device 130 arranged inside the cleaner 100 according to an embodiment of the disclosure.

The detection device 130 may be arranged between the suction device 120 and the suction part 110. For example, referring to FIG. 3, the suction device 120 may include a plurality of dust collecting units (a first dust collecting unit and a second dust collecting unit). Here, the dust collecting units may be provided to a cyclone dust collecting unit. The cyclone dust collecting unit generates a swirling air current and separates air and foreign substances by a centrifugal force. The air from which the foreign substances were separated may be discharged to the outside of the dust collecting units, and the foreign substances may be accumulated in the dust collecting units. When foreign substances are accumulated inside the dust collecting units to some degree, the user may separate the dust collecting units and discard the foreign substances in their insides.

The first dust collecting unit may be connected with the other end of the suction pipe, and separate big dust inside the air suctioned through the suction pipe. Here, a first filter may be arranged on one side surface of the first dust collecting unit, and filter small dust that was not separated from the first dust collecting unit. Also, the second dust collecting unit may be connected to the first filter, and separate dust in a fine size that was not separated (or was not filtered) from the first dust collecting unit and the first filter. The suction device 120 may prevent the dust suctioned through the cleaner 100 from being re-discharged to the air (or to the outside of the cleaner 100) based on the plurality of dust collecting units and the filter. Also, the suction device 120 may prevent breakdown of the motor by preventing suctioning of dust inside the motor arranged on the rear end of the first filter.

Meanwhile, to the second dust collecting unit, the aforementioned detection device 130 may be connected. That is, the detection device 130 may detect allergen substances that exist inside the air filtered through the first and second dust collecting units and the first filter. Through this, the detection device 130 may identify whether allergen substances exist inside the air suctioned in real time during a cleaning operation of the cleaner 100.

The air suctioned into the detection device 130 may be discharged from the detection device 130 and introduced into the cleaner 100 through a second filter (e.g., a motor filter) and a third filter. Here, the second filter may filter finer dust that was not filtered by the second dust collecting unit, and the third filter may filter ultra-fine dust in a size from 0.5 to 4.2 μm. Through this, the suction device 120 may prevent suctioning of the dust into the motor.

Meanwhile, FIG. 3 illustrates that the detection device 130 is arranged inside the suction device 120 according to an embodiment of the disclosure, and thus the disclosure is not limited thereto. That is, the detection device 130 may be installed inside the flow path of the air (or the dust) suctioned through the suction part 110, or may be installed to be connected with the suction part 110. That is, the detection device 130 may be located in various locations inside the cleaner 100 depending on embodiments.

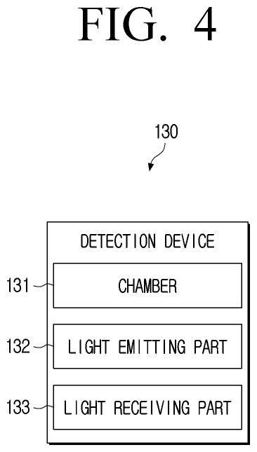

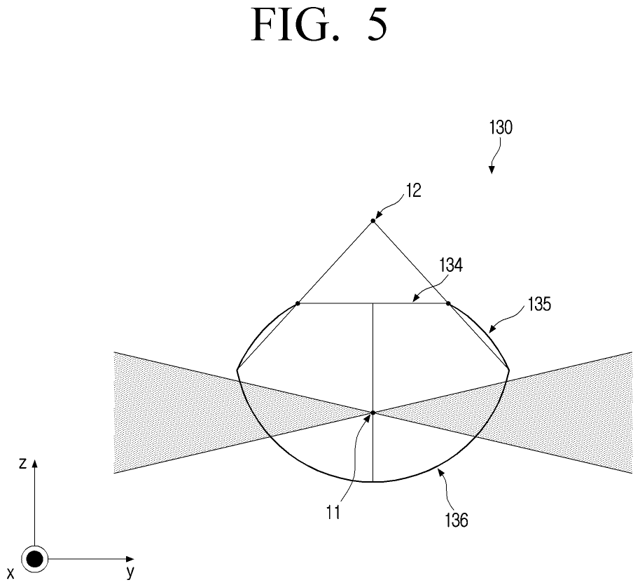

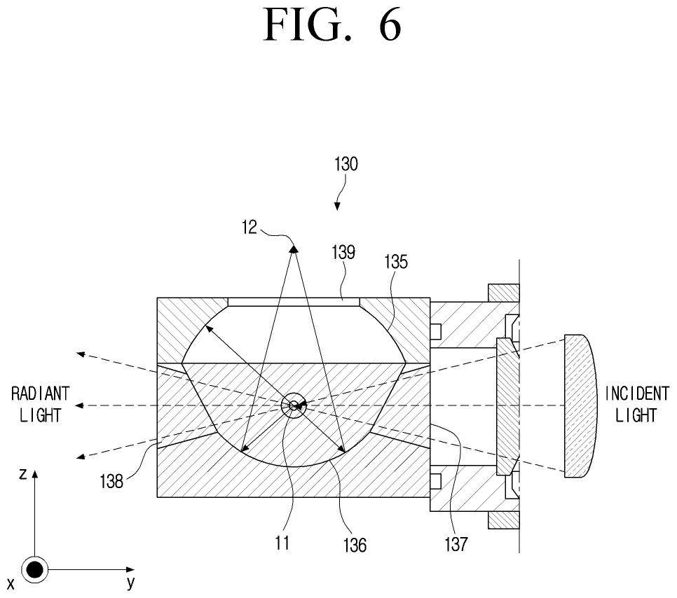

FIG. 4 is a schematic block diagram of the detection device 130 according to an embodiment of the disclosure. FIG. 5 is a cross-sectional view illustrating a chamber 131 of the detection device 130 according to an embodiment of the disclosure, and FIG. 6 is a diagram illustrating a relation between a circular mirror 135 and an elliptical mirror 136 inside the chamber 131 of the detection device 130 according to an embodiment of the disclosure.

Referring to FIG. 4, the detection device 130 includes a chamber 131, a light emitting part 132, and a light receiving part 133.

Referring to FIG. 4 and FIG. 5, the detection device 130 according to the embodiments of the disclosure includes a chamber 131 providing an introduction space for the air (or the dust) introduced into the detection device 130, a light emitting part 132 irradiating a light on the introduction space, and a light receiving part 133 detecting a scattering light and a fluorescence light injected from the introduction space in its inside.

The introduction space provided by the chamber 131 may be constituted in a form wherein a part of the circular mirror 135 which is the upper part and a part of the elliptical mirror 136 which is the lower part are coupled, and in a part or the entire parts of the inner wall of the introduction space, a mirror surface, i.e., the circular mirror 135 or the elliptical mirror 136 are arranged, and to the inside of the introduction space, air and dust are introduced through an inlet. Here, referring to FIG. 3 to FIG. 6, air and dust may be introduced into the introduction space of the detection device 130 from the +x axis direction.

Also, to the inlet of the chamber 131, an introduction nozzle of a nozzle part (not shown) may be connected, and air to be measured may be introduced into the introduction space through the introduction nozzle, and the introduction location of the air may be the part of a first focus 11 on which an incident light of the light emitting part 132 is focused.

Although not illustrated in the drawings, on the chamber 131 on the opposite side of the inlet, an air discharge hole may be provided, and the introduced air may be discharged to the outside of the detection device 130 (e.g., the suction pipe or the second filter of the suction device 120).

The center of the circular mirror 135 inside the introduction space is the first focus 11 of the elliptical mirror 136, and on the circular mirror 135, an opening is formed so as to correspond to a first light outlet 138.

Depending on embodiments, it is preferable that the radius of the circular mirror 135 is selected from values between 13 mm and 14 mm, and it is preferable that the size of the opening formed on the circular mirror 135 is selected from values between 14.5 mm and 15.5 mm.

The elliptical mirror 136 inside the introduction space may have two focuses, and a light that entered from the light emitting part 132 is substantially converged on the first focus 11 of the elliptical mirror 136 and is irradiated on the air introduced through the suction part 110, and the light that collided with the allergen substances in the air is scattered and refracted, and is injected to the outside of the introduction space toward the second focus 12 of the elliptical mirror 136 through the opening of the circular mirror 135 coupled with the elliptical mirror 136 by the elliptical mirror 136. Meanwhile, the outside of the introduction space is distinguished from the outside of the detection device 130. On the outside of the introduction space, the light receiving part 133 is arranged, and receives the light that was scattered and refracted or a fluorescence light emitted from the allergen substances.

Depending on embodiments, it is preferable that the length of the short axis of the elliptical mirror 136 is selected from values between 25.5 mm and 26.5 mm, and it is preferable that the length of the long axis of the elliptical mirror 136 is selected from values between 32 mm and 33 mm. Also, it is preferable that the distance between the first focus 11 and the second focus 12 of the elliptical mirror 136 is selected from values between 19 mm and 20 mm.

Referring to FIG. 4, a light that entered the light entrance 137 is substantially converged on the first focus 11 of the elliptical mirror 136 and is irradiated on the air introduced into the detection device 130, and the light that collided with the allergen substances in the air is scattered and refracted. The light refracted to the elliptical mirror 136 among the lights that were scattered and refracted is injected to the outside of the introduction space toward the second focus 12 through the first light outlet 138 by the elliptical mirror 136.

The light refracted to the circular mirror 135 among the lights that were scattered and refracted gets to be toward the first focus 11 of the elliptical mirror 136 by the circular mirror 135, and the light that passed through the first focus 11 of the elliptical mirror 136 is injected to the outside of the introduction space toward the second focus 12 through the first light outlet 138 by the elliptical mirror 136.

In case the introduction space was constituted as a part of the circular mirror 135 which is the upper part and a part of the elliptical mirror 136 which is the lower part were coupled, a light that entered by reflections of two times at the maximum is injected to the outside of the introduction space toward the second focus 12 through the first light outlet 138 by the elliptical mirror 136, and signal attenuation is in proportion to the number of being reflected on the reflector, and thus the detection device 130 according to the embodiments of the disclosure may minimize signal attenuation by reflection.

Explaining about reflections of two times at the maximum in more detail, in case the reflector inside the introduction space consists only of the elliptical mirror 136, the light refracted to the direction of the second focus 12 of the elliptical mirror 136 among the lights that were scattered and refracted is reflected by the elliptical mirror 136 and passes through the second focus 12, and the light that passed through the second focus 12 is reflected to the first focus 11 again by the elliptical mirror 136.

Here, the light refracted to the first focus 11 by the elliptical mirror 136 passes through the first focus 11, and the light that passed through the first focus 11 is reflected to the second focus 12 again by the elliptical mirror 136, and the light that passed through the second focus 12 is finally injected to the outside of the introduction space through the light outlet formed in the upper part of the elliptical mirror 136.

That is, the light refracted in the direction of the second focus 12 of the elliptical mirror 136 is injected to the outside through the light outlet formed in the upper part of the elliptical mirror 136 by reflections of at least three times. Thus, compared with the detection device 130 according to the embodiments of the disclosure, in case the reflector consists only of the elliptical mirror 136, it can be deemed that a signal attenuated by reflection of the reflector is much bigger.

In contrast, in the case of the detection device 130 according to the embodiments of the disclosure, the light refracted in the direction of the second focus 12 of the elliptical mirror 136 among the lights that were scattered and refracted is reflected by the circular mirror 135 and is reflected to the first focus 11 of the elliptical mirror 136 which is the center of the circular mirror 135, and the light that passed through the first focus 11 is injected to the outside of the introduction space toward the second focus 12 through the first light outlet 138 by the elliptical mirror 136.

That is, the light refracted in the direction of the second focus 12 of the elliptical mirror 136 is injected to the outside through the first light outlet 138 formed in the upper part of the circular mirror 135 by reflections of two times at the maximum, and thus the detection device 130 according to the embodiments of the disclosure can minimize signal attenuation by reflection of the reflector.

Also, comparing a case wherein the introduction space is constituted as a part of the circular mirror 135 which is the upper part and a part of the elliptical mirror 136 which is the lower part are coupled, and a case wherein the introduction space consists only of the elliptical mirror 136, in the case wherein the introduction space is constituted as a part of the circular mirror 135 which is the upper part and a part of the elliptical mirror 136 which is the lower part are coupled, the size of the introduction space is relatively smaller. Accordingly, the detection device 130 according to an embodiment of the disclosure can be miniaturized when compared with the detection device 130 wherein the introduction space consists only of the elliptical mirror 136, and by virtue of this, the detection device 130 can be arranged inside the cleaner 100.

The light emitting part 132 irradiating a light on the introduction space irradiates a UV light using light emitting diodes (LEDs) on a measurement sample inside the introduction space through the light entrance 137, and the irradiated UV light collides with the allergen substances inside the air introduced into the introduction space, and generates a scattering light and a fluorescence light.

Here, the LEDs which are light emitting elements of the light emitting part 132 may be arranged to irradiate a light in the direction of being introduced into the introduction space, i.e., the direction of the light entrance 137, and may emit a light of a UV area from 266 nm to 405 nm. Preferably, the LEDs may emit a light of a UV area from 340 nm to 380 nm, and may thereby minimize fluorescence of substances other than allergen substances in the air.

The light emitting part 132 collects the light irradiated into the introduction space to a designated point inside the introduction space. The designated point inside the introduction space means the first focus 11 of the elliptical mirror 136, and in the part of the first focus 11, the air suctioned through the suction part 110 is introduced inside the detection device 130, and thus the light focus of the light emitting part 132 and the introduction location of the suctioned air coincide, and accordingly, the ability of the detection device of detecting allergen substances can be improved.

Meanwhile, the light receiving part 133 that detects a scattering light and a fluorescence light injected from the first light outlet 138 of the introduction space may include a fluorescence light receiving part 133 and a scattering light receiving part 133.

The fluorescence light receiving part 133 and the scattering light receiving part 133 respectively receive a scattering light and a fluorescence light injected to the outside of the introduction space and generate detection signals for the received lights, and transmit the signals to the signal processing part (not shown).

In the case of autofluorescence by allergen substances, the signal is a very fine signal compared to a scattering light, and thus the fluorescence light receiving part 133 may be implemented as a photo multiplier tube (PMT), and the detected fluorescence may include information on whether microorganism exist and their amount.

The scattering light receiving part 133 may be implemented as a photodiode, and the scattering light detected by the scattering light receiving part 133 may include information on whether fine dust exists in the suctioned air and its amount.

Here, the detection device 130 may transmit the signals detected from the fluorescence light receiving part 133 and the scattering light receiving part 133 to the signal processing part, and calculate whether fine dust and microorganisms exist and their amount according to a predetermined algorithm.

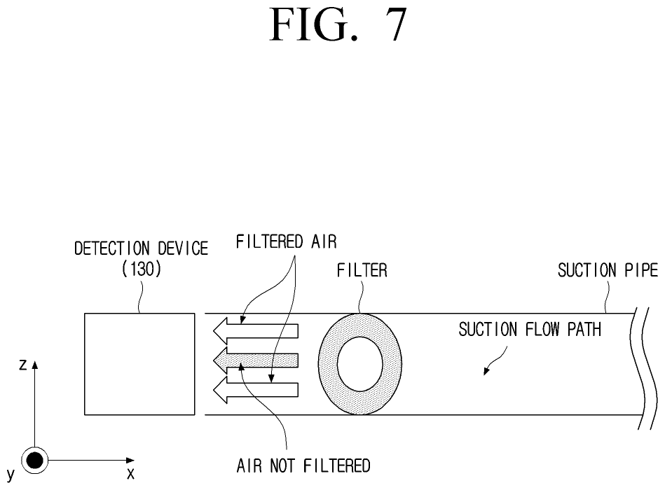

FIG. 7 is a diagram illustrating a form of instruction of air suctioned into the detection device 130 according to an embodiment of the disclosure.

Meanwhile, the suction pipe connected with the detection device 130 may include a filter that includes a hole in the center. Specifically, the filter may include a plurality of radiation holes, and include a first hole having a bigger diameter than the radiation holes in the center of the filter. Here, the first hole may be formed inside the filter so as to correspond to the center area of the flow channel formed by the suction pipe. Also, the first hole may be formed inside the filter so as to correspond to the first focus 11 inside the introduction space. Accordingly, the filter may be implemented as a form of a donut.

Meanwhile, the form of air suctioned into the introduction space of the detection device 130 by the filter may be in a form wherein air from which dust was filtered as it passed through the plurality of radiation holes of the filter encloses air from which dust was not filtered as it passed through the first hole of the filter. By virtue of this, in the case of the air introduced into the introduction space of the detection device 130, air including dust can be prevented from contacting the reflector inside the chamber 131, the light emitting part 132, and the light receiving part 133. In particular, as air including dust is concentrated in the area of the first focus 11, allergen substances inside the dust can be identified more correctly.

The at least one processor 140 according to an embodiment of the disclosure is electronically connected with the suction device 120 and the detection device 130, and controls the overall operations and functions of the cleaner 100.

The at least one processor 140 may include one or more of a central processing unit (CPU), a graphics processing unit (GPU), an accelerated processing unit (APU), a many integrated core (MIC), a digital signal processor (DSP), a neural processing unit (NPU), a hardware accelerator, or a machine learning accelerator. The at least one processor 140 may control one or a random combination of the other components of the electronic device, and perform an operation related to communication or data processing. Also, the at least one processor 140 may execute one or more programs or instructions stored in the memory. For example, the at least one processor 140 may perform the method according to an embodiment of the disclosure by executing one or more instructions stored in the memory.

In case the method according to an embodiment of the disclosure includes a plurality of operations, the plurality of operations may be performed by one processor 140, or performed by a plurality of processors. For example, when a first operation, a second operation, and a third operation are performed by the method according to an embodiment, all of the first operation, the second operation, and the third operation may be performed by a first processor, or the first operation and the second operation may be performed by the first processor (e.g., a generic-purpose processor), and the third operation may be performed by a second processor 140 (e.g., an artificial intelligence-dedicated processor).

The at least one processor 140 may be implemented as a single core processor including one core, or it may be implemented as one or more multicore processors including a plurality of cores (e.g., multicores of the same kind or multicores of different kinds). In case the at least one processor 140 is implemented as multicore processors, each of the plurality of cores included in the multicore processors may include an internal memory of the processor 140 such as a cache memory, an on-chip memory, etc., and a common cache shared by the plurality of cores may be included in the multicore processors. Also, each of the plurality of cores (or some of the plurality of cores) included in the multicore processors may independently read a program instruction for implementing the method according to an embodiment of the disclosure and perform the instruction, or the plurality of entire cores (or some of the cores) may be linked with one another, and read a program instruction for implementing the method according to an embodiment of the disclosure and perform the instruction.

In case the method according to an embodiment of the disclosure includes a plurality of operations, the plurality of operations may be performed by one core among the plurality of cores included in the multicore processors, or they may be implemented by the plurality of cores. For example, when the first operation, the second operation, and the third operation are performed by the method according to an embodiment, all of the first operation, the second operation, and the third operation may be performed by a first core included in the multicore processors, or the first operation and the second operation may be performed by the first core included in the multicore processors, and the third operation may be performed by a second core included in the multicore processors.

In the embodiments of the disclosure, the processor 140 may mean a system on chip (SoC) wherein at least one processor and other electronic components are integrated, a single core processor, a multicore processor, or a core included in the single core processor or the multicore processor. Also, here, the core may be implemented as a CPU, a GPU, an APU, a MIC, a DSP, an NPU, a hardware accelerator, or a machine learning accelerator, etc., but the embodiments of the disclosure are not limited thereto.

Hereinafter, the at least one processor 140 will be referred to as the processor 140, for the convenience of explanation.

The processor 140 controls the detection device 130 to emit a light of a predetermined wavelength while the suction device 120 is operating, and identifies whether allergen substances exist in the dust suctioned through the suction part 110 based on a wavelength of a light detected at the detection device 130.

Specifically, the processor 140 may control the light emitting part 132 of the detection device 130, and emit a light of a predetermined wavelength toward the air introduced into the introduction space inside the detection device 130. Here, the emitted light may be emitted toward the first focus 11 inside the introduction space. As explanation in this regard was described above, detailed explanation will be omitted.

In particular, when the suction device 120 is driven according to a user's control instruction or a predetermined condition, the processor 140 may control the light emitting part 132 and emit a light of the predetermined wavelength. Through this, while cleaning proceeds, the processor 140 may identify allergen substances inside the air (and the dust) suctioned into the cleaner 100 in real time, and identify the location and the area wherein allergen substances exist in a home wherein the cleaner 100 is located. As an example, if it is identified that the motor of the suction device 120 operates (e.g., rotates), the processor 140 may control the light emitting part 132 of the detection device 130, and emit a light of the predetermined wavelength to the introduction space of the detection device 130.

Meanwhile, the processor 140 may detect whether allergen substances exist inside the suctioned dust based on a wavelength detected at the detection device 130. Specifically, the processor 140 may detect a light emitted from the introduction space of the detection device 130 through the detection device 130. Here, the light emitted from the introduction space may include the light that was emitted by the light emitting part 132 and scattered by dust, etc. and collected through the light receiving part 133. Also, the light emitted from the introduction space may include the light that was emitted by the allergen substances existing in the dust after the allergen substances absorbed the light emitted by the light emitting part 132.

Here, the light emitted by the allergen substances may have a different wavelength from the light emitted by the light emitting part 132. As an example, the light emitting part 132 may emit a light having a wavelength from 350 nm to 405 nm. Also, the wavelength of the light emitted by the allergen substances may be from 220 nm to 500 nm. Accordingly, if the wavelength of the light collected at the light receiving part 133 is from 220 nm to 500 nm, the processor 140 may identify that allergen substances exist in the dust.

In other words, the processor 140 may collect lights of wavelengths in various sizes through the light receiving part 133. As described above, the processor 140 may receive a light of a wavelength in a predetermined size (a wavelength from 350 nm to 405 nm) that was reflected by dust, etc. and scattered. Alternatively, in case a light emitted from the light emitting part 132 was scattered by the reflector without collision with dust, allergen substances, etc., the processor 140 may receive a light of a wavelength in the predetermined size (a wavelength from 350 nm to 405 nm). In particular, in case there are allergen substances in the dust, the processor 140 may absorb a light that the allergen substances emit (i.e., a light of a wavelength from 220 nm to 500 nm) after absorbing the light emitted by the light emitting part 132. Here, the processor 140 may identify the size of the wavelength of the received light, and if it is identified that the identified size of the wavelength is from 220 nm to 500 nm, the processor 140 may identify that allergen substances exist in the suctioned dust.

Meanwhile, according to an embodiment of the disclosure, the processor 140 may identify the amount of allergen substances based on the strength of a detected light. Here, the processor 140 may identify the concentration level of allergen substances corresponding to the identified amount of the allergen substances, and control the suction device 120 to generate an airflow in suction strength corresponding to the identified concentration level.

Specifically, the processor 140 may identify the strength of a light having a wavelength greater than or equal to 220 nm and smaller than or equal to 500 nm among the lights detected through the light receiving part 133. As described above, a light of a wavelength greater than or equal to 220 nm and smaller than or equal to 500 nm is emitted by allergen substances that absorbed the lights emitted from the light emitting part 132, and accordingly, as there are more allergen substances in the dust, the strength of the light having a wavelength greater than or equal to 220 nm and smaller than or equal to 500 nm will increase. For this, the light receiving part 133 (more specifically, the fluorescence light receiving part 133 included in the light receiving part 133) may include an optoelectronic amplifier tube and a photodiode.

Meanwhile, the processor 140 may identify the concentration of allergen substances by dividing the concentration in predetermined levels based on the strength of the light having a wavelength greater than or equal to 220 nm and smaller than or equal to 500 nm. For example, if the strength of the light having a wavelength greater than or equal to 220 nm and smaller than or equal to 500 nm is greater than or equal to 0, and is smaller than or equal to a first threshold value, the processor 140 may identify the concentration of the allergen substances as a first level. Meanwhile, if the strength of the light having a wavelength greater than or equal to 220 nm and smaller than or equal to 500 nm is greater than or equal to the first threshold value, and is smaller than or equal to a second threshold value (the second threshold value is bigger than the first threshold value), the processor 140 may identify the concentration of the allergen substances as a second level. Also, if the strength of the light having a wavelength greater than or equal to 220 nm and smaller than or equal to 500 nm is greater than or equal to the second threshold value, the processor 140 may identify the concentration of the allergen substances as a third level. That is, as the concentration of the allergen substances is higher, the processor 140 may identify that the level is higher.

Meanwhile, this is merely an example, and the processor 140 may identify the concentration of allergen substances by applying various threshold values (or reference values) regarding the concentration of the allergen substances and dividing the concentration into levels in various numbers. However, hereinafter, it will be explained that the levels of the concentration of allergen substances are divided into three levels of the first to third levels, for the convenience of explanation of the disclosure.

Meanwhile, the processor 140 may identify the suction strength corresponding to the identified concentration level. That is, the processor 140 may drive the suction device 120 to suction dust in the strength corresponding to the concentration level of the allergen substances. Specifically, the processor 140 may rotate the motor inside the suction device 120 at the speed corresponding to the concentration level of the allergen substances. For example, in case the suction strength of the cleaner 100 is set as three stages (strong, medium, weak), the processor 140 may control the suction device 120 to suction dust in the strength corresponding to the concentration level of the allergen substances. Specifically, while the cleaner 100 is performing a cleaning operation with the suction strength set as “weak,” if the concentration of the allergen substances detected in the suctioned dust is identified as the second level, the processor 140 may change the suction strength from “weak” to “medium” to correspond to the concentration of the allergen substances (i.e., the second level) without the user's control instruction.

As described above, the cleaner 100 according to an embodiment of the disclosure can appropriately remove allergen substances in a home by detecting allergen substances in the dust in real time, identifying the concentration of the allergen substances, and then adjusting the suction strength according to the concentration of the allergen substances.

Meanwhile, as an example, the processor 140 may identify the type of a surface to be cleaned, and if the surface is identified as a surface to be cleaned wherein it is advantageous for allergen substances to propagate (or there is a high possibility that allergen substances may exist), the processor 140 may control the suction device 120 such that the suction strength of the cleaner 100 increases. Specifically, the processor 140 may identify the frictional force between the suction brush and the bottom surface included in the suction part 110, and identify the material of the bottom surface based on the identified frictional force.

For example, if the material of the bottom surface is identified as a type wherein there is a high possibility that allergen substances may exist such as a carpet, etc., the processor 140 may control the suction device 120 such that the suction strength of the cleaner 100 increases. Meanwhile, for identifying the type of the surface to be cleaned, i.e., the material of the bottom surface, the processor 140 may use a sensor connected to the brush or a camera installed in the suction part 110. That is, the processor 140 may acquire a sensing value regarding the moving direction and the moving speed, etc. of the brush by using the sensor, and identify the material of the bottom surface based on the acquired sensing value. Also, the processor 140 may identify the material of the bottom surface based on an image of the bottom surface acquired by using the camera.

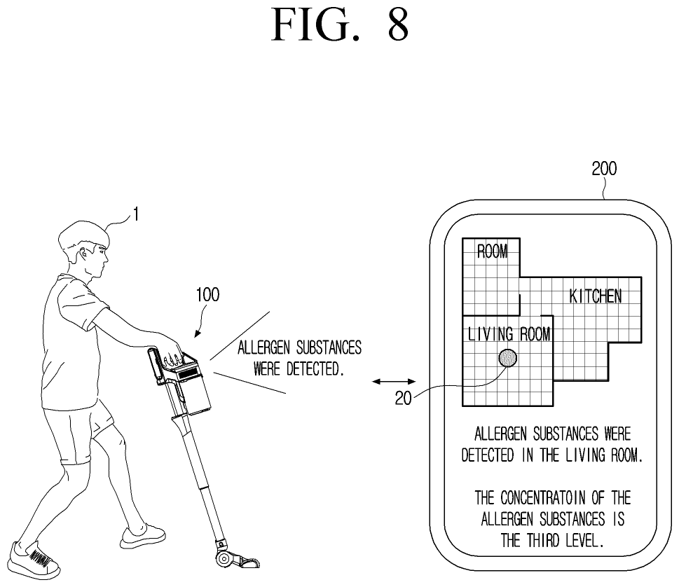

FIG. 8 is an exemplary diagram illustrating transmission of information regarding allergen substances to a user terminal through a communication interface according to an embodiment of the disclosure.

Meanwhile, the processor 140 may transmit information indicating that allergen substances were detected and information indicating the concentration of the identified allergen substances to a user terminal 200 through a communication interface (not shown) of the cleaner 100. As an example, the processor 140 may display map data corresponding to the home wherein the cleaner 100 is located and the location (or the place, the area, etc.) wherein allergen substances were detected on the map data, and transmit it to the user terminal 200 through the communication interface.

Alternatively, the processor 140 may transmit information on coordinates wherein allergen substances were detected on the map data to the user terminal 200 through the communication interface. By this, on the user terminal 200, the location wherein the allergen substances were detected on the map data may be displayed in real time. In addition, a message indicating that allergen substances were detected and a message indicating the concentration level of the allergen substances may be displayed together.

Meanwhile, according to an embodiment of the disclosure, the processor 140 may identify the amount of allergen substances identified in a plurality of areas of the space wherein the cleaner 100 is located based on the amount of the allergen substances, display information on the amount of the allergen substances corresponding to each area on the map data and store it in the memory, and periodically update the stored map data based on the amount of the allergen substances. Then, the processor 140 may transmit the updated map data to a user terminal device through the communication interface.

Specifically, in the memory of the cleaner 100, map data corresponding to the space wherein the cleaner 100 is located may be stored. The processor 140 may load the map data stored in the memory whenever the cleaner 100 performs a cleaning operation, and then update information on allergen substances on the map data. Here, the information on allergen substances may include information on the location wherein the allergen substances were detected in the map data, and information on the concentration of the allergen substances.

The processor 140 may identify the location of the area corresponding to the location wherein the allergen substances were detected on the map data, and display the concentration information of the allergen substances in the identified location of the area. Here, the concentration information of the allergen substances may be displayed in various forms such as a graphic object, a UI, etc. As an example, referring to FIG. 8, the processor 140 may change the color of the graphic object 20 to a color set to correspond to the concentration of the allergen substances, and display the object on the map data.

Meanwhile, the processor 140 may generate history information regarding the allergen substances for each area included in the space wherein the cleaner 100 is located by periodically updating allergen substances on the map data. Through this, the processor 140 may predict time points when allergen substances are detected, and also predict the concentration of allergen substances that are detected on each time point when allergen substances are detected.

In particular, the processor 140 may output warning information of allergen substances on a time point (or a time, a season, etc.) when allergen substances are predicted to be detected through a display (not shown) or an output interface (not shown), and output information (e.g., a message, etc.) requesting the user to operate the cleaner 100. For example, if it is identified, based on history information, that allergen substances are detected in March, and in particular, if it is identified that the concentration of allergen substances is high in the living room area in the space wherein the cleaner 100 is located, the processor 140 may output a message requesting performing of intensive cleaning for the living room area together with a warning message regarding allergen substances in a voice form through a speaker included in the output interface in March.

Also, in case the cleaner 100 is implemented as the robot cleaner 100 according to an embodiment of the disclosure, the cleaner 100 may automatically perform a cleaning operation for a place wherein it is identified that allergen substances would be detected or it is identified that the concentration of allergen substances would be high based on the history information. Explaining again based on the aforementioned example, if it is identified, based on history information, that allergen substances are detected in March, and in particular, if it is identified that the concentration of allergen substances is high in the living room area in the space wherein the cleaner 100 is located, the processor 140 may control the driving part and thereby control the robot cleaner 100 to travel in the living room area. Also, while the robot cleaner 100 travels in the living room area, the processor 140 may control the suction device 120 of the cleaner 100, and thereby control such that the cleaner 100 suctions the dust inside the living room area, in particular, allergen substances included in the dust.

Meanwhile, the processor 140 may also output information indicating that allergen substances were detected and information indicating the concentration of the identified allergen substances through the display (not shown) or the output interface (not shown) of the cleaner 100.

In this regard, according to an embodiment of the disclosure, the cleaner 100 may further include an output interface including a plurality of light emitting elements. The output interface including the plurality of light emitting elements may be arranged in the suction part 110. The processor 140 may transmit a driving current to the plurality of light emitting elements included in the output interface, and emit a light through the plurality of light emitting elements. Through this, while performing a cleaning operation, the processor 140 may make the plurality of light emitting elements emit a light, and thereby make dust, foreign substances, etc. existing on the surface to be cleaned observed more clearly. Meanwhile, although not illustrated in the drawings clearly, the cleaner 100 may further include a driver IC for driving the plurality of light emitting elements, and a driving circuit connected to each light emitting element.

Meanwhile, the plurality of light emitting elements may be implemented as a plurality of LED elements. In particular, as the plurality of LED elements, a plurality of red LED elements, a plurality of green LED elements, and a plurality of blue LED elements arranged in an array form may be included. Here, the processor 140 may change the color of the output light by adjusting the driving current applied to the red LED elements, the green LED elements, and the blue LED elements.

As an example, the processor 140 may control the plurality of light emitting elements such that a light of a color corresponding to the concentration level of the identified allergen substances is emitted. Through this, while the user performs cleaning by using the cleaner 100, the processor 140 may recognize the allergen substances existing on the surface to be cleaned and the concentration of the allergen substances based on the color of the light output from the output interface.

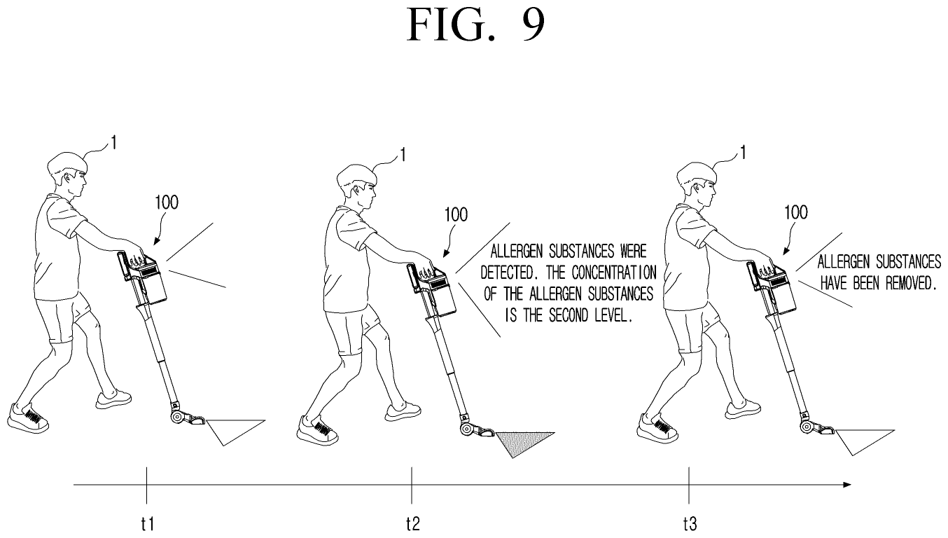

FIG. 9 is an exemplary diagram illustrating control of a plurality of light emitting elements such that a light of a color corresponding to the concentration of allergen substances is emitted according to an embodiment of the disclosure.

Referring to FIG. 9, in case the concentration of allergen substances is divided into three concentration levels (i.e., the first to third levels), a green light may be set in case allergen substances are not detected, a yellow light may be set in case the concentration level of allergen substances is the first level, an orange light may be set in case the concentration level of allergen substances is the second level, and a red light may be set in case the concentration level of allergen substances is the third level, respectively. The processor 140 may control the output interface to emit a green light on a t1 time point when allergen substances are not detected. As described above, the processor 140 may apply a driving current only to the green LED elements among the red LED elements, the green LED elements, and the blue LED elements, and thereby control such that the output interface emits a green light.

Here, while the user cleans another surface to be cleaned different from the t1 time point on the t2 time point, if allergen substances are detected, and it is identified that the concentration level of the detected allergen substances is the second level, the processor 140 may control the output interface to emit an orange light through the output interface. As described above, the processor 140 may adjust the driving current applied to the red LED elements, the green LED elements, and the blue LED elements, and change the color of the light emitted by the output interface from green to orange.

Also, the processor 140 may identify that the concentration of the detected allergen substances is reduced as the allergen substances that existed on the surface to be cleaned are suctioned through the cleaner 100. Accordingly, the processor 140 may change the color of the light emitted through the output interface from orange to yellow, and green, in a corresponding manner to the reduced concentration of the allergen substances.

According to an embodiment of the disclosure, the cleaner 100 may further include a collection part collecting the suctioned dust which includes at least one heating coil. The collection part may be a dust collecting unit wherein the suctioned dust that passed through the suction part 110, the suction device 120, and the detection device 130 is finally collected. For example, the collection part may be implemented as a dust can that is attached and connected to one side of the cleaner 100.

Here, the collection part may include at least one heating coil. In particular, the at least one heating coil may be attached on one surface inside the collection part, and if power is applied to the at least one heating coil, heat emitted by the at least one heating coil may be transmitted to the inside of the collection part.

If the amount of allergen substances is greater than or equal to a predetermined value, the processor 140 may provide power to the at least one heating coil such that the allergen substances are inactivated. Here, the amount of the allergen substances may be the amount of the suctioned allergen substances identified by the processor 140 in real time, or the total amount of the allergen substances detected through the detection device 130 after the cleaner 100 performed a cleaning operation.

In particular, the processor 140 may identify the total amount of the allergen substances suctioned into the cleaner 100 during a predetermined time after the cleaner 100 performed the cleaning operation as the total amount of the allergen substances accumulated in the collection part. Here, if the total amount of the allergen substances is greater than or equal to the predetermined value, the processor 140 may provide power to the at least one heating coil. Here, when power is provided to the at least one heating coil, the temperature inside the collection part may increase by the heat emitted by the at least one heating coil. Through this, the allergen substances accumulated inside the collection part may be inactivated.

However, the disclosure is not limited thereto, and the processor 140 may continuously apply power to the at least one heating coil while the cleaner 100 performs cleaning.

Also, on one side surface of the collection part, a filter may be included. Here, the filter included in the collection part may be implemented in the form of mesh including holes of a predetermined size through which allergen substances can be filtered. As allergen substances are filtered by the filter, dust, etc. excluding the allergen substances may be collected inside the collection part.

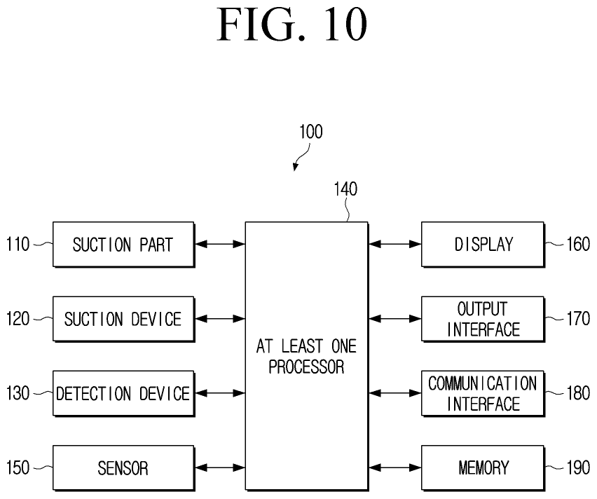

FIG. 10 is a detailed configuration diagram of the cleaner 100 according to one or more embodiments of the disclosure.

The cleaner 100 includes a suction part 110, a suction device 120, a detection device 130, a sensor 150, a display 160, an output interface 170, a communication interface 180, a memory 190, and at least one processor 140. Among the components illustrated in FIG. 10, regarding components that are overlappingly illustrated in FIG. 2, detailed explanation will be omitted.

The sensor 150 includes a temperature sensor 150. The processor 140 may measure the temperature/humidity in the surrounding environment of the cleaner 100 by using the temperature/humidity sensor 150, and if it is identified that the measured temperature/humidity coincide with the temperature/humidity at which allergen substances are activated (or increase) identified based on the history information, the processor 140 may output information requesting to operate the cleaner 100 to the user.

Meanwhile, the sensor 150 may include a gyro sensor 150 and an acceleration sensor 150. The sensor 150 may detect a movement of the cleaner 100, and acquire data regarding the movement of the cleaner 100. Specifically, the gyro sensor 150 may detect a rotation of the cleaner 100 and acquire angular velocity data for the cleaner 100, and the acceleration sensor 150 may detect a movement of the cleaner 100 and acquire acceleration data for the cleaner 100.

Also, the sensor 150 may include a LiDAR sensor 150 and a Time of Flight (ToF) sensor 150. The LiDAR sensor 150 and the ToF sensor 150 may detect an object around the cleaner 100, and acquire distance information between the cleaner 100 and the object. Here, the processor 140 may generate map data regarding the space wherein the cleaner 100 is located based on the acquired distance information. For this, the processor 140 may use an SLAM algorithm stored in the memory 190.

The display 160 outputs various kinds of visual information regarding the cleaner 100. Specifically, the display 160 may not only display a GUI related to control of the cleaner 100, but also display information on allergen substances. As an example, the display 160 may display information regarding whether allergen substances are detected, and the concentration of the allergen substances. In particular, in case allergen substances are detected in real time, the display 160 displays a warning message regarding the allergen substances to the user, and thereby enables the user to correctly identify the place and the location wherein allergen substances exist in the home.

For this, the display 160 may be implemented as a display 160 including self-emitting elements or a display 160 including non-self-emitting elements and a backlight. For example, the display 160 may be implemented as displays 160 in various forms such as a liquid crystal display (LCD), an organic light emitting diodes (OLED) display 160, light emitting diodes (LED), micro LED, mini LED, a plasma display panel (PDP), a quantum dot (QD) display 160, quantum dot light emitting diodes (QLED), etc.

In the display 160, driving circuits that may be implemented in forms such as an a-si TFT, a low temperature poly silicon (LTPS) TFT, an organic TFT (OTFT), etc., a backlight unit, etc. may also be included. Meanwhile, the display 160 may be implemented as a touch screen combined with a touch sensor 150, a flexible display 160, a rollable display 160, a 3D display 160, a display 160 to which a plurality of display 160 modules are physically connected, etc.

Also, the display 160 may constitute a touch screen together with a touch panel. Here, the display 160 may perform functions not only as the output interface 170 outputting visual information, but also an input interface.

The output interface 170 may output information acquired by the cleaner 100 to the outside. For example, the output interface 170 may be implemented as a speaker. The speaker may output a voice message indicating that allergen substances were detected, and a voice message indicating the concentration of the allergen substances.

The communication interface 180 may include various communication modules for performing communication between the cleaner 100 and an external device. As an example, the communication interface 180 may include a wireless communication module, and may include, for example, a cellular communication module using at least one of 3^{rd} Generation (3G), 3^{rd} Generation Partnership Project (3GPP), Long Term Evolution (LTE), LTE Advanced (LTE-A), a code division multiple access (CDMA), a wideband CDMA (WCDMA), a universal mobile telecommunications system (UMTS), Wireless Broadband (WiBro), or a Global System for Mobile Communications (GSM), etc. As another example, a wireless communication module may include, for example, at least one of wireless fidelity (WiFi), Bluetooth, Bluetooth low energy (BLE), or Zigbee.

In case allergen substances are detected, the communication interface 180 may transmit information indicating whether allergen substances were detected and information indicating the concentration of the allergen substances to the user terminal 200 registered in the cleaner 100. In particular, the communication interface 180 may display the area wherein the allergen substances were detected on the map data, and then transmit the map data to the user terminal device.

The memory 190 may store an operating system (O/S) for driving the cleaner 100. Also, in the memory 190, various kinds of software programs or applications for the cleaner 100 to operate according to the various embodiments of the disclosure may be stored. In addition, in the memory 190, various kinds of information such as various kinds of data, etc. that is input or set, or generated during execution of a program or an application may be stored. Further, in the memory 190, various kinds of software modules for the cleaner 100 to operate according to the various embodiments of the disclosure may be included, and the processor 140 may perform the operations of the cleaner 100 according to the various embodiments of the disclosure by executing the various kinds of software modules stored in the memory 190. For this, the memory 190 may include a semiconductor memory 190 such as a flash memory 190, or a magnetic storage medium such as a hard disk, etc.

In particular, in the memory 190, map data corresponding to the space wherein the cleaner 100 is located may be stored. Here, the processor 140 may periodically update information on allergen substances on the map data whenever the cleaner 100 performs a cleaning operation or allergen substances are detected, and then store the updated map data in the memory 190. Then, based on the updated map data, the processor 140 may generate history information regarding allergen substances, and then store the information in the memory 190. Here, the history information regarding allergen substances may include information on the time points when allergen substances for the plurality of areas inside the space wherein the cleaner 100 is located are generated, change of the concentration of the allergen substances according to time, etc.

The memory 190 may be implemented in the form of a memory 190 embedded in the cleaner 100, or in the form of a memory that can be attached to or detached from the cleaner 100, according to the usage of stored data. For example, in the case of data for operating the cleaner 100, the data may be stored in a memory 190 embedded in the cleaner 100, and in the case of data for an extended function of the cleaner 100, the data may be stored in a memory 190 that can be attached to or detached from the cleaner 100.

Meanwhile, in the case of a memory 190 embedded in the cleaner 100, the memory 190 may be implemented as at least one of a volatile memory 190 (e.g.: a dynamic RAM (DRAM), a static RAM (SRAM), or a synchronous dynamic RAM (SDRAM), etc.) or a non-volatile memory 190 (e.g.: an one time programmable ROM (OTPROM), a programmable ROM (PROM), an erasable and programmable ROM (EPROM), an electrically erasable and programmable ROM (EEPROM), a mask ROM, a flash ROM, a flash memory 190 (e.g.: NAND flash or NOR flash, etc.), a hard drive or a solid state drive (SSD)).

Also, in the case of a memory 190 that can be attached to or detached from the cleaner 100, the memory 190 may be implemented in forms such as a memory 190 card (e.g., compact flash (CF), secure digital (SD), micro secure digital (Micro-SD), mini secure digital (Mini-SD), extreme digital (xD), a multi-media card (MMC), etc.), an external memory 190 that can be connected to a USB port (e.g., a USB memory 190), etc.

According to an embodiment, the memory 190 may store information on a plurality of neural network (or, artificial intelligence) models. Here, the feature of storing information on neural network models may mean storing various kinds of information related to the operations of the neural network models, for example, information on at least one layer included in a neural network model, information on parameters, biases, etc. used in each of the at least one layer, etc. Meanwhile, it is obvious that, depending on implementation examples of the processor 140, information on neural network models can be stored in the internal memory 190 of the processor 140. For example, in case the processor 140 is implemented as dedicated hardware, information on neural network models may be stored in the internal memory 190 of the processor 140.

Other than the above, the cleaner 100 may further include a user interface. The user interface is a component that is used for the cleaner 100 to perform interactions with the user, and the processor 140 may receive inputs of various kinds of information such as control information of the cleaner 100, etc. through the user interface. Meanwhile, the user interface may include at least one of a touch sensor 150, a motion sensor 150, a button, a jog dial, a switch, or a microphone, but is not limited thereto.

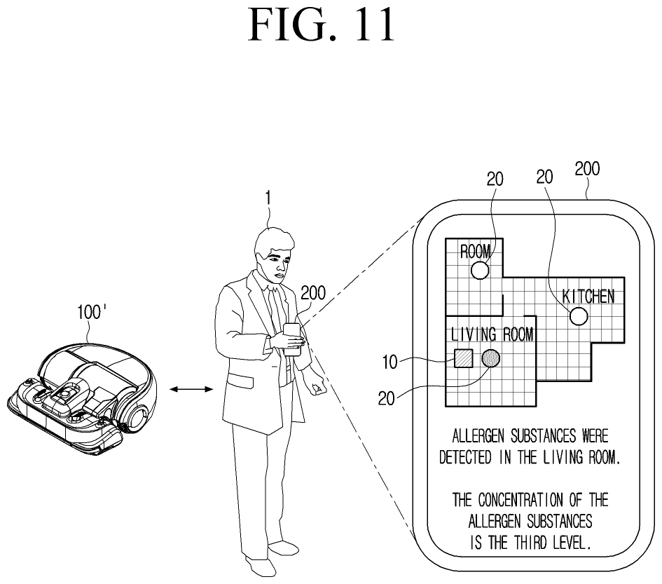

FIG. 11 is an exemplary diagram illustrating transmission by a robot cleaner of information regarding allergen substances to a user terminal through a communication interface according to an embodiment of the disclosure.

Meanwhile, as described above, the cleaner 100 according to an embodiment of the disclosure may also be implemented as a robot cleaner.

Here, the robot cleaner 100′ may perform a cleaning operation while travelling along a predetermined travelling path in the space wherein the robot cleaner 100′ is located. More specifically, the processor 140 may acquire information on obstacles, walls, objects, etc. (e.g., point cloud data, etc.) in the space wherein the robot cleaner 100′ is located through a sensor, and then generate map data corresponding to the space wherein the robot cleaner 100′ is located based on the acquired information. Here, the processor 140 may generate the map data corresponding to the space wherein the robot cleaner 100′ is located based on a simultaneous localization and mapping (SLAM) algorithm stored in the memory 190. Then, the processor 140 may store the generated map data in the memory 190.

Then, when the processor 140 receives an input of the user's control instruction for operating the robot cleaner 100′, the processor 140 may set the travelling path of the robot cleaner 100′ on the stored map data, and perform an operation corresponding to the control instruction (e.g., a cleaning operation, a mopping operation, etc.) according to the set travelling path. Meanwhile, the travelling path may be set based on the area wherein it is set that the robot cleaner 100′ performs cleaning among the areas dividing the space wherein the robot cleaner 100′ is located, the location of the area, etc.

In the case of the robot cleaner 100′, the aforementioned embodiments of the disclosure can be applied identically. More specifically, referring to FIG. 11, in case the robot cleaner 100′ performs cleaning in the home according to the user's control instruction, the robot cleaner 100′ may perform a cleaning operation for a plurality of areas included in the home, and at the same time, identify whether allergen substances exist in each area or identify the amount of the allergen substances existing in each area. Here, an allergen detecting operation of the robot cleaner 100′ may be performed according to a separate control instruction (i.e., a control instruction requesting to detect allergen substances), or if a control instruction for a cleaning operation is input (or is received), an operation of detecting allergen substances may be additionally performed at the same time as the cleaning operation.

Meanwhile, the processor 140 may generate information on allergen substances, and transmit the information to the user terminal 200.

As an example, the processor 140 may transmit coordinate information wherein allergen substances were detected on the map data corresponding to the home wherein the robot cleaner 100′ is located to the user terminal 200 through the communication interface. By this, on the user terminal 200, the location wherein allergen substances were detected on the map data may be displayed in real time. In addition, on the user terminal 200, a graphic object indicating the concentration of the allergen substances, a message indicating that allergen substances were detected, and a message indicating the concentration level of the allergen substances may be displayed together.

Specifically, referring to FIG. 11, the processor 140 may transmit real time location information of the robot cleaner 100′, information on whether allergen substances were detected, and information on the concentration of the allergen substances to the user terminal 200 through the communication interface 180. Here, on the user terminal 200, a graphic object 10 indicating the real time location of the robot cleaner 100′ on the map data and a graphic object 20 indicating the concentration of the allergen substances regarding the area wherein the robot cleaner 100′ performed cleaning may be displayed together. Through this, the user may monitor information on allergen substances in the home in real time even in a space separated from the robot cleaner 100′.

In particular, the processor 140 may set the color of the graphic object 20 differently according to the concentration of the allergen substances, and transmit color information of the graphic object together with the concentration information of the allergen substances detected in each area to the user terminal 200. Accordingly, the user terminal 200 may display the graphic object 20 having the color corresponding to the concentration of the allergen substances detected in each area on the map data.

Also, the processor 140 may identify the concentration level of the allergen substances corresponding to the amount of the detected allergen substances, and control the suction device 120 of the robot cleaner 100′ to generate an airflow in the suction strength corresponding to the identified concentration level.

In addition, the processor 140 may output a voice message indicating that allergen substances were detected through the output interface 170 (e.g., a speaker).

Further, the processor 140 may control a plurality of light emitting elements arranged on one surface of a suction hole of the robot cleaner 100′ such that a light of a color corresponding to the concentration level of the allergen substances is emitted.

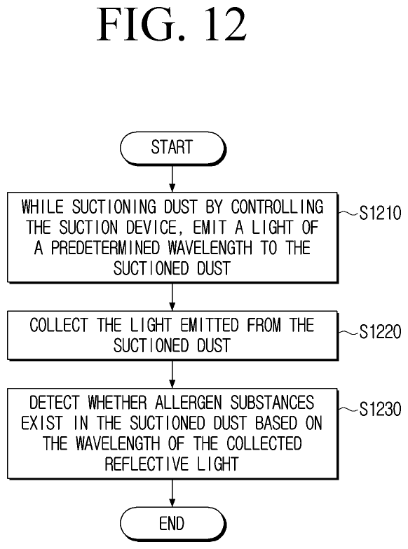

FIG. 12 is a flow chart schematically illustrating a controlling method of the cleaner 100 according to an embodiment of the disclosure.

First, the processor 140 controls the suction dust 120 and suctions the dust. Then, the processor 140 emits a light of a predetermined wavelength to the suctioned dust during the operation of the suction device 120 in operation S1210, and collects the light emitted from the suctioned dust in operation S1220. Here, the predetermined wavelength may be from 350 nm to 405 nm.

The processor 140 detects whether allergen substances exist in the suctioned dust based on the collected light in operation S1230. In particular, the processor 140 detects whether allergen substances exist in the suctioned dust based on the wavelength of the collected reflective light in operation S1130. If the wavelength of the reflective light is from 220 nm to 500 nm, the processor 140 may identify that allergen substances exist in the dust.

Here, the processor 140 may identify the strength of the reflective light, and identify the amount of the allergen substances based on the identified strength of the reflective light. Then, the processor 140 may identify a concentration level of the allergen substances corresponding to the identified amount of the allergen substances, and control the suction device 120 to generate an airflow in suction strength corresponding to the identified concentration level.

Also, the processor 140 may control a plurality of light emitting elements included in the output interface 170 such that a light of a color corresponding to the identified concentration level is emitted.

Meanwhile, the processor 140 may identify the amount of allergen substances identified in a plurality of areas of the space wherein the cleaner 100 is located based on the amount of the allergen substances, display information on the amount of the allergen substances corresponding to each area on map data corresponding to the space and store it in the memory 190. Then, the processor 140 may identify the amount of the allergen substances whenever the suction device 120 operates, periodically update the stored map data based on the identified amount of the allergen substances, and transmit the updated map data to a user terminal device.

Also, if the amount of the allergen substances is greater than or equal to a predetermined value, the processor 140 may provide power to at least one heating coil included in a collection part such that the allergen substances are inactivated.

Meanwhile, methods according to the aforementioned various embodiments of the disclosure may be implemented in forms of applications that can be installed on a conventional cleaner. Alternatively, the methods according to the aforementioned various embodiments of the disclosure may be performed through a trained neural network based on deep learning (or a neural network that performed deep learning), i.e., a learning network model. Also, the methods according to the aforementioned various embodiments of the disclosure may be implemented just with software upgrade, or hardware upgrade for a conventional cleaner. In addition, the aforementioned various embodiments of the disclosure can also be performed through an embedded server provided on a cleaner, or an external server of a cleaner.

Meanwhile, according to an embodiment of the disclosure, the aforementioned various embodiments may be implemented as software including instructions stored in machine-readable storage media, which can be read by machines (e.g.: computers). The machines refer to devices that call instructions stored in a storage medium, and can operate according to the called instructions, and the devices may include a display device according to the embodiments disclosed herein (e.g.: a display device A). In case an instruction is executed by a processor, the processor may perform a function corresponding to the instruction by itself, or by using other components under its control. An instruction may include a code that is generated or executed by a compiler or an interpreter. A storage medium that is readable by machines may be provided in the form of a non-transitory storage medium. Here, the term ‘non-transitory’ only means that a storage medium does not include signals, and is tangible, but does not indicate whether data is stored in the storage medium semi-permanently or temporarily.

Also, according to an embodiment, the methods according to the aforementioned various embodiments may be provided while being included in a computer program product. A computer program product refers to a product, and it can be traded between a seller and a buyer. A computer program product can be distributed on-line in the form of a storage medium that is readable by machines (e.g.: a compact disc read only memory (CD-ROM)), or through an application store (e.g.: Play Store™). In the case of on-line distribution, at least a portion of a computer program product may be stored in a storage medium such as the server of the manufacturer, the server of the application store, and the memory of the relay server at least temporarily, or may be generated temporarily.

Further, each of the components according to the aforementioned various embodiments (e.g.: a module or a program) may consist of a singular object or a plurality of objects. Also, among the aforementioned corresponding sub components, some sub components may be omitted, or other sub components may be further included in the various embodiments. Alternatively or additionally, some components (e.g.: a module or a program) may be integrated as an object, and perform the functions that were performed by each of the components before integration identically or in a similar manner. A module, a program, or operations performed by other components according to the various embodiments may be executed sequentially, in parallel, repetitively, or heuristically. Or, at least some of the operations may be executed in a different order or omitted, or other operations may be added.

Also, while preferred embodiments of the disclosure have been shown and described, the disclosure is not limited to the aforementioned specific embodiments, and it is apparent that various modifications may be made by those having ordinary skill in the technical field to which the disclosure belongs, without departing from the gist of the disclosure as claimed by the appended claims. Further, it is intended that such modifications are not to be interpreted independently from the technical idea or prospect of the disclosure.

What is claimed is:

1. A cleaner comprising:
a suction part;
a suction device which generates airflow such that dust is suctioned through the suction part;
a detection device which is arranged on one side of the suction part, and emits a light of a predetermined wavelength, and detects a wavelength of a partial light emitted; and
at least one processor which controls the suction device,
wherein the at least one processor is configured to:
control the detection device to emit a light of a predetermined wavelength during an operation of the suction device, and
detect whether allergen substances exist in the dust suctioned through the suction part based on a wavelength of a light reflected from the dust.

2. The cleaner of claim 1,
wherein the at least one processor is configured to:
identify the amount of the allergen substances based on the strength of the detected light,
identify a concentration level of the allergen substances corresponding to the identified amount of the allergen substances, and
control the suction device to generate the airflow in suction strength corresponding to the identified concentration level.

3. The cleaner of claim 2,
wherein the cleaner further comprises:
an output interface including a plurality of light emitting elements, and
the at least one processor is configured to:
control the plurality of light emitting elements such that a light of a color corresponding to the identified concentration level is emitted.

4. The cleaner of claim 2,
wherein the cleaner further comprises:
a communication interface; and
a memory storing map data corresponding to a space wherein the cleaner is located, and
the at least one processor is configured to:
identify the amount of allergen substances identified in a plurality of areas of the space based on the amount of the allergen substances,
display information on the amount of the allergen substances corresponding to each area on the map data and store it in the memory,
periodically update the stored map data based on the amount of the allergen substances, and
transmit the updated map data to a user terminal device through the communication interface.

5. The cleaner of claim 2, further comprising:
a collection part collecting the suctioned dust which includes at least one heating coil,
wherein the at least one processor is configured to:
based on the amount of the allergen substances being greater than or equal to a predetermined value, provide power to the at least one heating coil such that the allergen substances are inactivated.

6. The cleaner of claim 1,
wherein the predetermined wavelength is from 350 nm to 405 nm, and
the at least one processor is configured to:
based on the wavelength of the reflected light being from 220 nm to 500 nm, identify that the allergen substances exist.

7. The cleaner of claim 1,
wherein the detection device comprises:
a chamber into which the dust is introduced and of which inside is implemented as a reflector;
a light entrance into which an incident light enters;
a chamber body including a first light outlet and a second light outlet for injecting an incident light irradiated on the dust;
a light emitting part which irradiates the incident light to the light entrance, and blocks ambient lights introduced into the incident light; and
a light receiving part which delivers a radiant light injected from the first light outlet by separating the light into a first path and a second path, detects a scattering light from the radiant light transmitted to the first path, and blocks the ambient lights introduced into the radiant light transmitted to the second path and detects the reflected light.

8. The cleaner of claim 7,
wherein the suction part comprises:
a suction pipe which provides a suction flow path of the suctioned dust, and
the suction pipe comprises:
a filter arranged on one end, and the filter comprises a first hole corresponding to the center area of the suction flow path, and
in the chamber,
dust filtered through the suction pipe is introduced.

9. A controlling method of a cleaner, the method comprising:
controlling the detection device to suction dust; and
detecting whether allergen substances exist in the suctioned dust,
wherein the suctioning comprises:
emitting a light of a predetermined wavelength to the suctioned dust during an operation of the suction device; and
collecting the light emitted from the suctioned dust, and
the detecting comprises:
detecting whether allergen substances exist in the suctioned dust based on a wavelength of the collected reflective light.

10. The controlling method of claim 9,
wherein the detecting comprises:
identifying the strength of the reflective light, and identifying the amount of the allergen substances based on the identified strength of the reflective light, and
the controlling method further comprises:
identifying a concentration level of the allergen substances corresponding to the identified amount of the allergen substances; and
controlling the suction device to generate an airflow in suction strength corresponding to the identified concentration level.

11. The controlling method of claim 10, further comprising:
controlling a plurality of light emitting elements such that a light of a color corresponding to the identified concentration level is emitted.

12. The controlling method of claim 10, comprising:
identifying the amount of allergen substances identified in a plurality of areas of a space wherein the cleaner is located based on the amount of the allergen substances;
displaying information on the amount of the allergen substances corresponding to each area on map data corresponding to the space and storing it in the memory;
identifying the amount of the allergen substances whenever the suction device operates, and periodically updating the stored map data based on the identified amount of the allergen substances; and
transmitting the updated map data to a user terminal device.

13. The controlling method of claim 10, comprising:
based on the amount of the allergen substances being greater than or equal to a predetermined value, providing power to at least one heating coil included in a collection part such that the allergen substances are inactivated.

14. The controlling method of claim 9,
wherein the predetermined wavelength is from 350 nm to 405 nm, and
the detecting comprises:
based on the wavelength of the reflected light being from 220 nm to 500 nm, identifying that the allergen substances exist.

15. The controlling method of claim 9,
wherein the cleaner comprises:
a detection device, and
the detection device comprises:
a chamber into which the dust is introduced and of which inside is implemented as a reflector;
a light entrance into which an incident light enters;
a chamber body including a first light outlet and a second light outlet for injecting an incident light irradiated on the dust;
a light emitting part which irradiates the incident light to the light entrance, and blocks ambient lights introduced into the incident light; and
a light receiving part which delivers a radiant light injected from the first light outlet by separating the light into a first path and a second path, detects a scattering light from the radiant light transmitted to the first path, and blocks the ambient lights introduced into the radiant light transmitted to the second path and detects the reflected light.
